(19) United States
(12) Patent Application Publication
(10) Pub. No.: US 2010/0044210 A1
(43) Pub. Date: Feb. 25, 2010

(54) METHOD OF DIGESTING CELLULOSE TO GLUCOSE USING SALTS AND MICROWAVE (muWAVE) ENERGY

(75) Inventor: J. Michael Robinson, Odessa, TX (US)

Correspondence Address: ROBERT W STROZIER, P.L.L.C, PO BOX 429, BELLAIRE, TX, 77402-0429, US

(73) Assignee: The Board of Regents of the University of Texas System, Austin, TX (US)

(21) Appl. No.: 12/195,376

(22) Filed: Aug. 20, 2008

(51) Int. Cl. C07H 1/00

(52) U.S. Cl. 204/157.68

(57) ABSTRACT

A method for digesting biomass carbohydrate polymers is disclosed. The method includes irradiating the polymers in an acid/salt solution in the presence of a microwave energy field, where the acid, salt and microwave energy are sufficient to convert a desired amount of the polymers into useful platform chemicals.

### GOVERNMENTAL RIGHTS

The present invention claims priority to and the benefit of U.S. Provisional Patent Application Ser. No. 60/956,851, filed Aug. 20, 2007.

### RELATED APPLICATIONS

Some or all of the subject matter disclosed in this application was funded to some degree by funds supplied by the United States Government under NSF grant no. 26-0705-0320.

### BACKGROUND OF THE INVENTION

1. Field of the Invention

The present invention relates to a method to hydrolyze naturally occurring carbohydrate polymers from biomass sources into several chemical platforms. Abundant biomass carbohydrates, hemi-celluloses, and cellulose are more difficult to hydrolyze than starch.

More particularly, the present invention relates to a method to hydrolyze naturally occurring carbohydrate polymers from biomass sources into several chemical platforms including the steps of reacting the polymers with microwave irradiation in a solution of very dilute salt water that is also very dilute acid. Sequential selective conditions allow complete separation of hemi-cellulose derived solutions of sugars, followed by subsequent selective procedures to gain high conversions of various platform chemicals, such as glucose or levulinic acid or hydroxymethylfurfural.

2. Description of the Related Art

Plant material wastes (biomass) are made up of five main components: cellulose, hemicellulose, lignin, crude protein and ash. Cellulose is generally a linear, unbranched glucose-based homopolymer, i.e., a polysaccharide, of relatively high molecular weight. Hemicellulose is typically a branched and/or unbranched polymer of D-glucose, D-mannose, L-arabinose and D-xylose of about 100-200 sugar residues per polymer chain. Lignins are amorphous crosslinked phenolic polymers that occur uniquely in vascular plants and comprise 20-30% of most wood.

Processing of biomass is important in several industries such as waste management, pulp and paper, food manufacture, and energy production among others. For example, it is known to hydrolyze cellulosic materials into monosaccharides for varying purposes including feed stocks for other chemicals, food stuffs, fuels, and the like. In addition, conversion of biomass to sugars usable directly as food or as chemical reagents is an interest in planning long-term space missions. In space, the fixation of carbon dioxide into edible biomass can be performed by agronomical species such as wheat, potatoes, soybeans, and the like. However, only half of the crop is edible. Of the inedible portion, approximately 50-68 percent is polysaccharide which can be reduced into fermentable sugars. The remainder is primarily unusable lignin. Waste paper produced in space is another source of usable polysaccharide since it is primarily cellulose.

Reduction of polysaccharides by hydrolysis is well known in the art. Two basic methods are generally used: (1) chemical treatment, e.g., reduction using an acid catalyst; and (2) biological breakdown using enzymes or microbes such as fungus. Such methods generally include one or more pretreatments to increase hydrolysis reaction rate and yield. Pretreatments typically increase the availability and surface area of reducible polysaccharides by disturbing the physical and molecular structure of the feed material and/or fractionating the lignocellulosic material into its lignin, hemicellulose and cellulose components.

Examples of common pretreatment techniques include milling and sizing; steam solubilizing in the presence of chemicals such as caustic widely used in pulp and paper manufacture, ammonia, chlorite, sulfur dioxide, amines, acids both dilute and concentrated, etc.; autohydrolysis by high temperature steam (i.e., 220° C.-275° C.); steam explosion (a forceful extrusion of a steam/feed mixture through an orifice by a sharp pressure drop across the orifice); and electron irradiation. In a given process, common pretreatments may be combined. Additional details surveying the prior pretreatment art are described in Petersen et al., The Engineering Society for Advancing Mobility Land Sea Air and Space (SAE International) technical paper 901282, Jul. 9-12, 1990.

The drawbacks of both of these types of processes are evident. The reaction rate of enzymatic hydrolysis is low and a significant concentration of undesirable byproducts such as furfural can result. Concentrated acid-catalyzed hydrolysis produces little or none of the byproducts seen in enzymatic hydrolysis and has a higher reaction rate, but acid consumption is high and product recovery from the reaction effluent is expensive. Health and environmental hazards are also present. Dilute acids can be used, but sugar degradation and yield reduction can, and usually do occur. Pretreatments in both these processes consume energy in the form of steam. Where chemicals are used, they must be removed from the end product. Even with the prior art pretreatment, enzymatic hydrolysis is relatively slow and undesired byproducts are still present.

In space missions, a unique set of constraints are presented. The expendable materials used in the process must either be carried as part of the mission payload or be produced on board the space habitat or vehicle. The availability of energy sources is likewise limited. While sulfuric acid is an excellent catalyst for the hydrolysis reaction, it is difficult to manufacture in space. Enzymes, on the other hand, can theoretically be made in space, but the primary problem of low yield remains. The constraints of space limit the suitability of pretreatments using steam and many chemicals since the preparation and recycle of the required chemicals is generally difficult. Steam production is also energy intensive and the furfural byproduct requires a relatively complex separation step.

J. Azuma et al., Journal of Fermentation Technology, Vol. 62, No. 4, pp 377-384 (1984), discloses a microwave radiation pretreatment method for enzymatic saccharification of lignocellulosic wastes. Enzymatic susceptibility of the wastes is said to be improved by aqueous microwave pretreatment above 160° C. with maximum improvement at 223° C.-228° C. Following pretreatment, maximum yield of reducing sugars is 77-84% of the polysaccharide present in the original lignocellulose waste.

P. J. Blotkamp et al., American Institute of Chemical Engineering (AIChE) Symposium Series No. 181, Vol. 74(1981), describes a simultaneous saccharification of cellulose and fermentation to ethanol utilizing enzymes of the mold Trichoderma reesea and the yeast Saccharomyces cerevisiae. The hydrolysis rate was said to increase when compared to saccharification alone due to removal of competitive inhibition by glucose and cellobiose.

U.S. Pat. No. 5,196,069 to Cullingford, et al. disclosed a low energy consumption, low complexity apparatus and method for the conversion of cellulosic wastes into soluble saccharides suitable for terrestrial or space use wherein the chemicals utilized are not extremely hazardous, easily recyclable and can be prepared in space. An apparatus and method produced soluble saccharides without unwanted decomposition byproducts such as furfural.

Thus, there is a need in the art for hydrolyzing biomass carbohydrate monomers, oligomers and/or polymers into useful platform chemicals such as glucose, furfurals, levulinic acid and other compounds or mixtures or combinations thereof.

### SUMMARY OF THE INVENTION

The present invention provides a method to hydrolyze naturally occurring carbohydrate polymers from biomass sources into several chemical platforms including the steps of reacting the polymers with microwave irradiation in a solution of very dilute salt water that is also very dilute acid. Sequential selective conditions allow complete separation of hemi-cellulose derived solutions of sugars, followed by subsequent selective procedures to gain high conversions of various platform chemicals, such as glucose or Levulinic acid or hydroxymethylfurfural.

The present invention also provides a products prepared by the methods of this invention.

The present invention also provides a method for producing a fuel comprising hydrolyzing biomass carbohydrate polymers into glucose and fermenting the glucose into a ethanol rich fuel.

The present invention also provides a method for producing a fuel comprising hydrolyzing biomass carbohydrate polymers into glucose and fermenting the glucose into a methanol and ethanol rich fuel.

The present invention is based on the discovery of a physicochemical pretreatment which greatly enhances the reaction rate and reduces the energy consumption of enzymatic hydrolysis of cellulosic waste materials. The method comprises irradiating an aqueous cellulose feed mixture with microwave energy in a reactor in the presence of hydrochloric acid (which can be easily produced from salt by electrochemical means) and a metal salt, while maintaining an advanced equilibrium pressure. The invention is also well suited for commercial and governmental uses to convert biomass into energy and useful platform chemicals. The invention is also well suited for use in space since energy usage is low and the concentration of chemicals utilized are not hazardous, they are easily recycled and can be prepared in space. The sugars recovered can be used directly as food or converted, e.g., via microbial action, into other types of food, into fuels (e.g., alcohols via fermentation) or other organic chemicals.

In one embodiment, the present invention provides a method for pretreating cellulose for enzymatic hydrolysis. The method comprises irradiating cellulose with microwaves at superatmospheric pressure in the presence of water, an acid and a metal salt effective to substantially enhance hydrolysis. The irradiating step is preferably maintained at an equilibrium pressure of from about 4 to about 10 atm. The invention uses dilute solutions of an acid and a metal salt.

In another embodiment, the present invention provides a method for converting cellulose into saccharides. The method comprises the steps of: irradiating cellulose in the presence of an aqueous acidic metal salt solution. In most embodiments, the acid is a mineral acid. In other embodiments, the acid is a purified hydrochloric acid. The reaction is generally carried out using microwaves at superatmospheric pressure for short reaction times to enhance the formation soluble saccharides. The irradiating step is maintained at an equilibrium pressure of from about 2 to about 20 atm, preferably from about 4 to about 10 atm. The method may further include, separating the acidic metal salt solution from the irradiated cellulose solids wherein the separated acidic metal salt solution may be recycled to the irradiating step, separating the saccharides and recycling to the hydrolyzing step and fermenting the saccharides into ethanol.

In a further embodiment, the present invention provides an apparatus for converting a cellulose into saccharides, comprising: an autoclave reactor having an agitator and a pressure controller; a first charge to the autoclave reactor comprising an aqueous mixture of cellulose and an acid; a microwave radiation source for energizing the cellulose; a hydrolysis vessel having temperature and pH control; a second charge to the hydrolysis vessel comprising an aqueous mixture of the irradiated cellulose and enzymes suitable for hydrolyzing the irradiated cellulose into soluble saccharides; and means for transferring the irradiated cellulose to the hydrolysis vessel.

### DESCRIPTION OF THE DRAWINGS

The invention can be better understood with reference to the following detailed description together with the appended illustrative drawings in which like elements are numbered the same.

FIG. 1 depicts conversions of cellulose with weak acids and microwave energy.

FIG. 2 depicts an NMR spectra of cellulose digestion products.

FIG. 3 depicts % conversion in the presence of microwave energy and weak organic acid (acetic acid—AcOH) with and without salt (NaCl).

FIG. 4 depicts effects of salt on the efficiency of microwave digestion of cellulose at 208° C. in phosphoric acid (H₃PO₄), sulfuric acid (H₂SO₄) and hydrochloric acid (HCl). In most embodiments, the acid is hydrochloric acid. In other embodiments, the acid is pure hydrochloric acid stored in plastic containers.

FIG. 5 depicts cellulose digestion at 170° C. in the presence of 1% acid calculated by classical Saeman's Kinetics.

FIG. 6 depicts cellulose conversion using microwave irradiation in the presence of sulfuric acid at about 160° C. or hydrochloride acid at 167° C. at different acid concentrations and reaction times.

FIG. 7 depicts cellulose conversion using microwave irradiation in the presence of 1% hydrochloride acid at 167° C. to glucose or levulinic acid.

FIG. 8A depicts selectivity to products vs. acid concentration of microwave digestion of cellulose in HCl—the circled conversion evidences a high conversion to 5-hydroxymethyl furfural.

FIG. 8B depicts salt concentration vs conversion of cellulose in 0.01 M HCl—the circled conversion evidences a high conversion to 5-hydroxymethyl furfural.

FIG. 9 depicts combined severity vs. conversion of cellulose using microwave irradiation in the presence of 0.01 M HCl and 0.05 M NaCl and selectivity to glucose, levulinic acid and 5-hydroxymethyl furfural.

FIG. 10 depicts cellulose conversion using microwave irradiation in the presence of hydrochloric acid and various metal salts to produce different product distributions.

FIG. 11 depicts fast microwave removal of hemicellulose, protein & minerals in the presence of 1% hydrochloric acid.

FIG. 12 depicts a reactor system of use in the present invention.

### DETAILED DESCRIPTION OF THE INVENTION

The inventor has found that efficient cellulose digestion can occur in the present of a dilute salt acid solution in the presence of a microwave energy field. The digestion can be tuned using different transition metal catalysts to product differing amount of certain platform chemicals.

A particularly important feature is the ability to use very dilute acid and very dilute salt water for these hydrolyses. The hydrolysis of hemicellulose takes place in minutes rather than hours. The hydrolysis of cellulose takes place again in minutes rather than hours. These studies show that the most difficult part of biomass carbohydrates to hydrolyze, the alpha-crystalline cellulose, is easily hydrolyzed under these conditions. These studies also show that it is very important as to what type and how much salt ions are in solution. Heavy metal salts or polyvalent cations generally induced much more decomposition to levulinic acid. Alkali or alkali earth type salts such as sodium chloride greatly assist hydrolysis to glucose solutions, but only in these very dilute concentrations. In acid solution of 1% concentration the salts have almost no effect. In a survey of most of the elements in the periodic chart, both copper and zinc chloride salts were found to be exceptionally useful. Copper chloride salts accelerate the desirable reaction to glucose versus decomposition products thus giving a very high ratio in just 2-3 minutes. Zinc chloride salts tend to slow the reaction, but allow a high ratio albeit only a fair conversion of the hydroxymethylfurfural to be formed from cellulose. Chromium (+2) chloride, greatly accelerates the hydrolysis, but also provides almost exclusively levulinic acid even without added HCl. In experiments with purified dry alpha-crystalline cellulose, such as Avicel, total conversions to soluble products reached only into the high 80% levels due to some polymer decomposition products. However, when these conditions were applied to fresh biomass (not dried) essentially total conversion of the carbohydrates was achieved. Two to three sequential hydrolyses for the cellulose fraction of biomass was effective in converting all that carbohydrate into predominantly glucose solutions using selected temperature and times with these dilute salt acid solutions.

Strong glucose concentrations in solution can be prepared in this manner after absorption removal of small amounts of impurities caused by slight or partial degradation of glucose to the hydroxymethylfurfural and subsequently conversion of hydroxymethylfurfural (HMF) to levulinic acid (LA). Likewise, purified hydroxymethylfurfural can be washed from the (commercially available) absorption medium to provide this separate platform chemical. Alternatively, more severe conditions of temperature and time will allow any or all of the carbohydrates to be converted into levulinic acid (LA) and the corresponding formic acid. These products can be simply separated by distillation.

Present technology cannot achieve strong glucose solutions with weak acid solutions. Strong glucose solutions are not only required for fermentation technology, but may be useful as a platform for other chemicals. No one, outside the inventor, has recognized the importance of the type or the extent of metal salts in such acid hydrolyzing solutions. Most of the industry today uses sulfuric acid rather than our preferred HCl. HCl, although more expensive, gives faster reaction and is indeed easier to control in the microwave reactor to the products as specified. Moreover, sulfuric acid tends to produce more decomposition products. Other acids, such as phosphoric or acetic, prove to be too weak to effect the reactions desired at reasonable temperatures and pressures and do not give good conversions.

One reference in the literature, Zuwei in 2001, used dilute HCl with dilute salt solutions, but did not know the temperature or pressure with his system, and he only used this to hydrolyze starch. Zuwei did not use this solution and microwave technology for treating cellulose, hemicellulose or any raw biomass, which is a combination of hemicellulose, cellulose, and/or lignin. Zuwei did not find that copper and zinc salts were particularly useful in short time degradation of hemicellulose, cellulose, and/or lignin. Zuwei did not find that chromium or other polyvalent heavy metals are particularly useful for degradation of glucose to levulinic acid in situ. The inventor has shown that it is also important not only to use pure acid, but also to store this dilute acid solution in plastic containers. If these solutions are stored in glass, various cationic salts are leached from the glass and these salts mixtures cause varying amounts of degradation depending on the glass chemistry.

This technology allows for several chemical platforms to be selectively created from the carbohydrates of biomass by fractionation. The cost of reagents to affect these transformations is greatly reduced because very dilute solutions acid and very dilute salt are used. The energy provided by microwave heating methods is also consumes a lot less energy than the conventional heating through the walls and the medium of a vessel. The technology of this invention may finally allow for cellulosic biomass processes for a modern biorefinery, rather than only using sugar-based or starch-based agricultural crops as feedstock for the ethanol fermentation processing. In this manner, waste crop materials such as corn stover or wood chips from managed forests are suitable feedstocks for this microwave hydrolysis processes of this invention. Thus, human and livestock food chains would not be impacted opening up many biomass resources for future fuel and as sources for platform chemicals.

Hydrolysis with dilute acid without salt is much less effective and correspondingly hydrolysis with salt but without trace amount of acid is not effective at the conditions of temperature and time used in this invention.

The objective of the present invention was to develop an efficient chemical method to produce several platform chemicals from biomass. The chemical process was designed to convert hemicellulose into xylose, glucose, or monosaccharides or mixtures thereof, and furfurals, and to convert cellulose into glucose, levulinic acid, hydroxymethylfurfural or mixtures thereof. One of the major problems faced by methods designed to digest cellulose is the presence of α-crystalline cellulose, which is notoriously difficult to hydrolyze prior to this invention. The use of very dilute acid (concentrations between about 0.005 M and about 0.025M) and very dilute metal salts (concentrations between about 0.01 and about 0.05 M) make the method of cellulose digestion of this invention well suited for downstream processing, because the low concentration of acid and metal salts often do not adversely affect downstream processing such as fermentation or product separation. The fermentation enzymes generally used can withstand the dilute acid and metal salt concentrations used in the present cellulose hydrolysis method without the need for rigorous neutralization and/or separations.

### EXPERIMENTS OF THE INVENTION

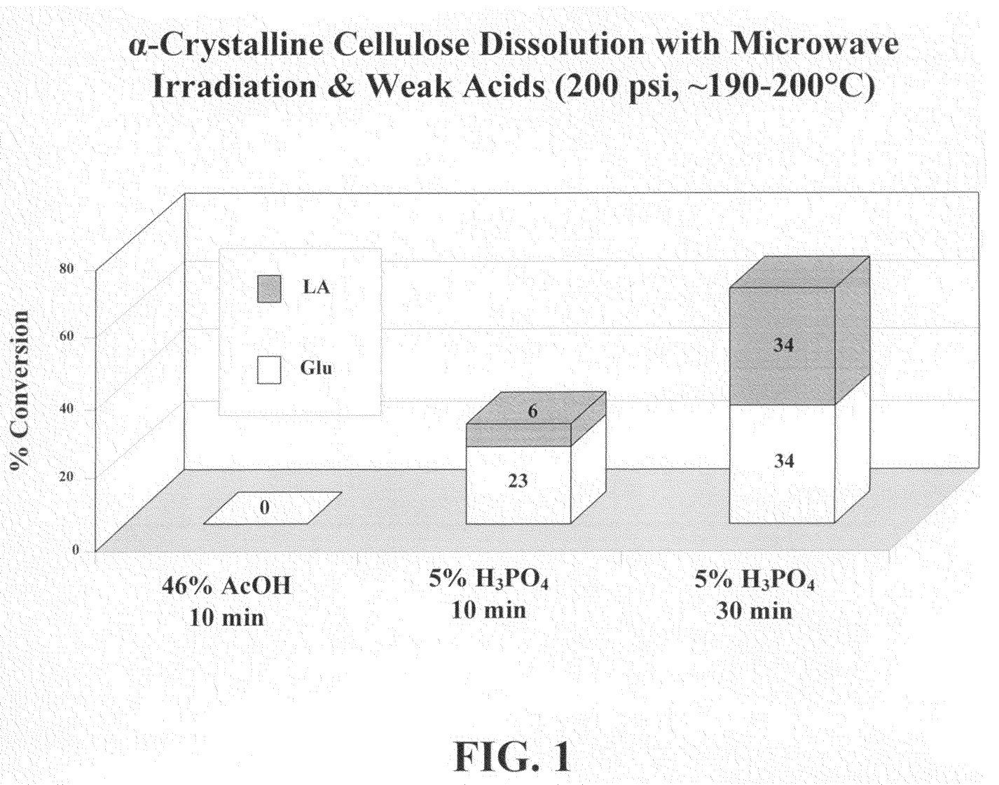

Referring to FIG. 1, α-crystalline cellulose dissolution using microwave energy and weak acids at about 200 psi in a temperature range between about 190° C. and about 200° C. is shown. Such treatment results in about an 80% conversion of α-crystalline cellulose into a mixture of glucose (Glu) and levulinic acid (LA) in acetic acid and phosphoric acid subject to different irradiation times.

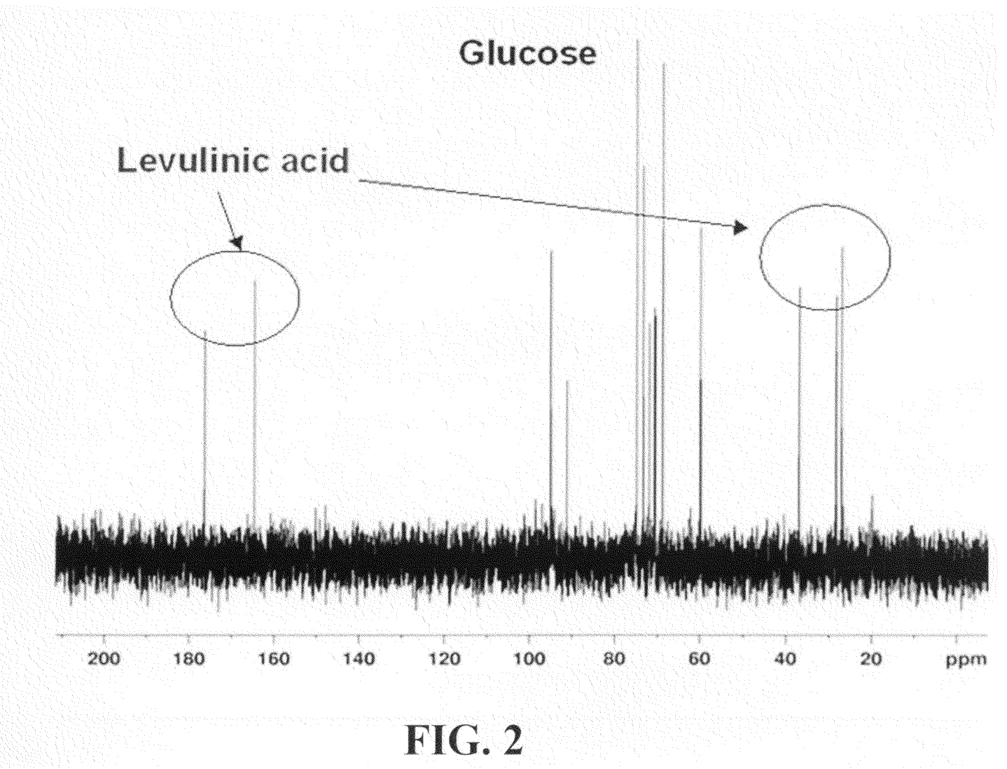

Referring to FIG. 2, a 13C NMR spectrum of a digested cellulose product is shown. The spectrum shown the absorptions for glucose and leuvulinic acid.

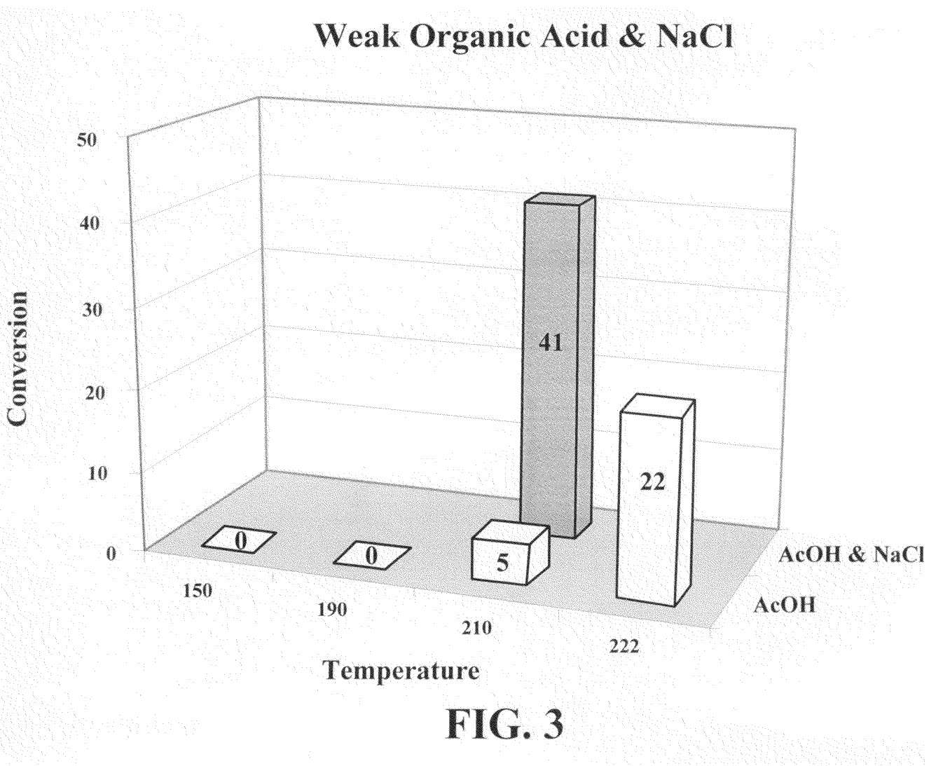

Referring to FIG. 3, the conversion of cellose in acetic acid with or without sodium chloride in the presence of microwave radiation is shown at temperatures between 150° C. and 222° C. The addition of sodium chloride switches the product from glucose to leuvulinic acid. Chen in 1996 reported the hydrolysis of starch in 5M HCl at 100° C. irradiated for 5 minutes with microwave (μW) radiation yielding 100% glucose.

Zuwei in 2001 reported the hydrolysis of starch in 0.01M HCl and 0.05M MClₓ for less than 10 minutes with microwave (μW) radiation yielding 100% glucose. However, Zuwei did not disclose the temperature or pressure of the reaction. Lee in 2006 reported the conversion of wood in 0.5-2% H₂SO₄ in less than 10 minutes with microwave (μW) radiation yielding 36% glucose with an 80% conversion of hemicellulose and a 60% conversion of cellulose.

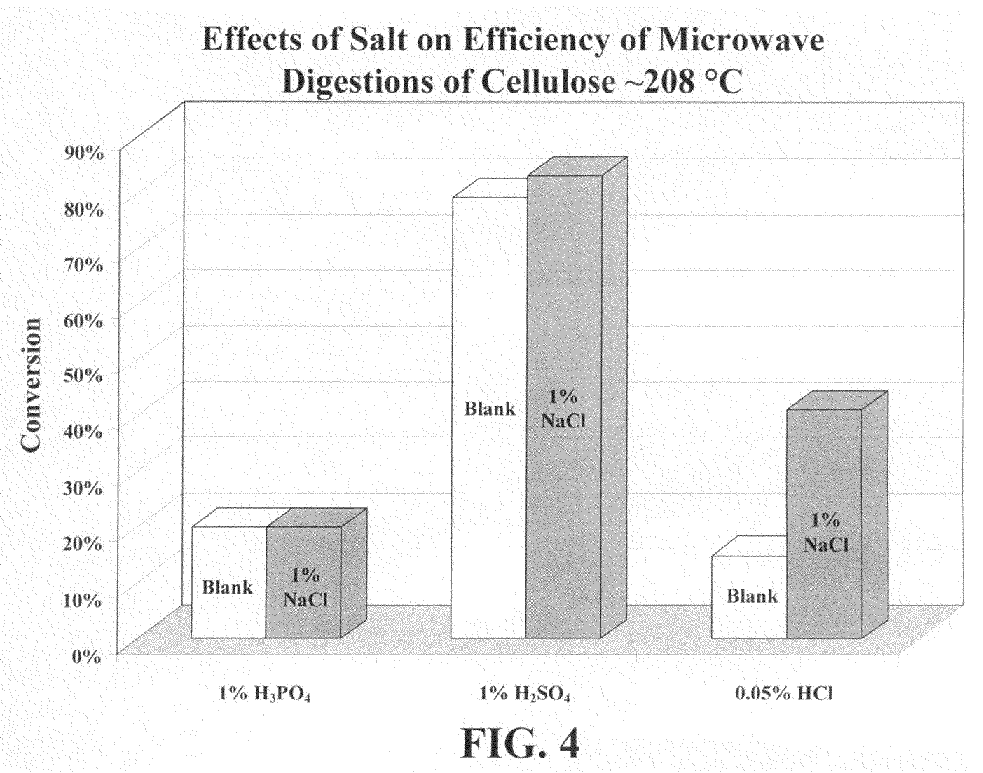

Referring to FIG. 4, the effects of added sodium chloride on microwave digestion of cellulose was studied in the presence of phosphoric acid (H₃PO₄), sulfuric acid (H₂SO₄) and hydrochloric acid (HCl). The data showed that the addition of salt made a significant difference in digestion using hydrochloride acid and had little effect on conversion for cellulose digestion using phosphoric acid or sulfuric acid.

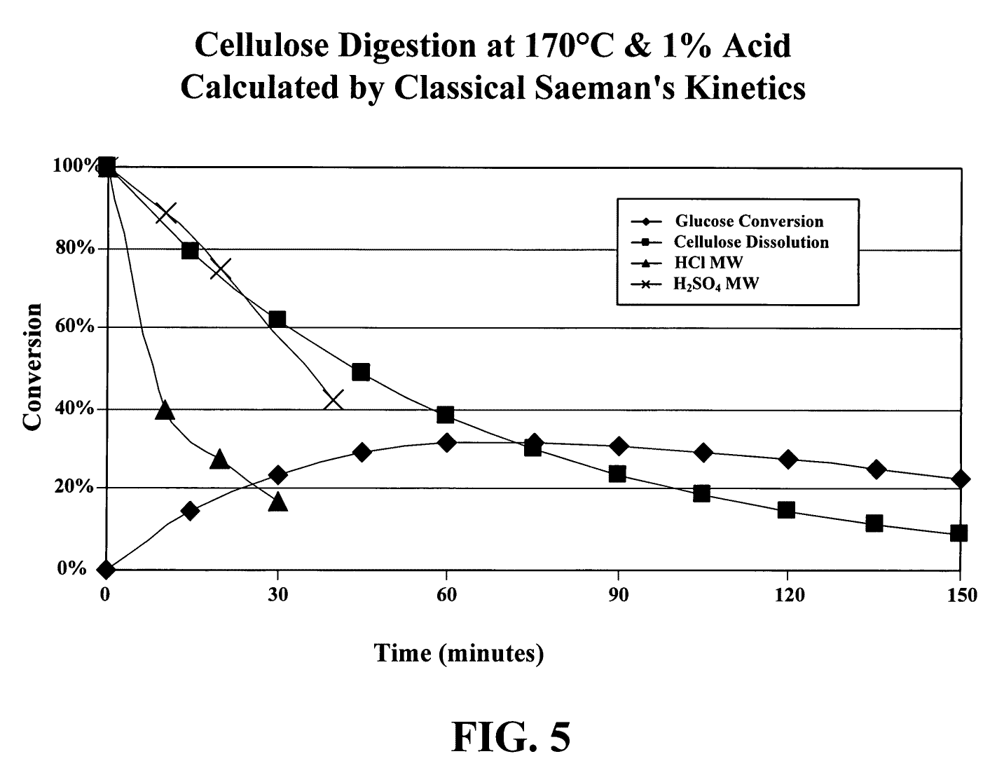

Referring to FIG. 5, the effects of cellulose conversion using microwave irradiation in the presence of sulfuric and hydrochloric acids was studied at 170° C. The conversion was analyzed using classical Saeman's kinetics. The data shows that at short times, glucose conversion and cellulose dissolution are more rapidly varying than at long duration. The data also shows that the acids are consumed quickly at short times.

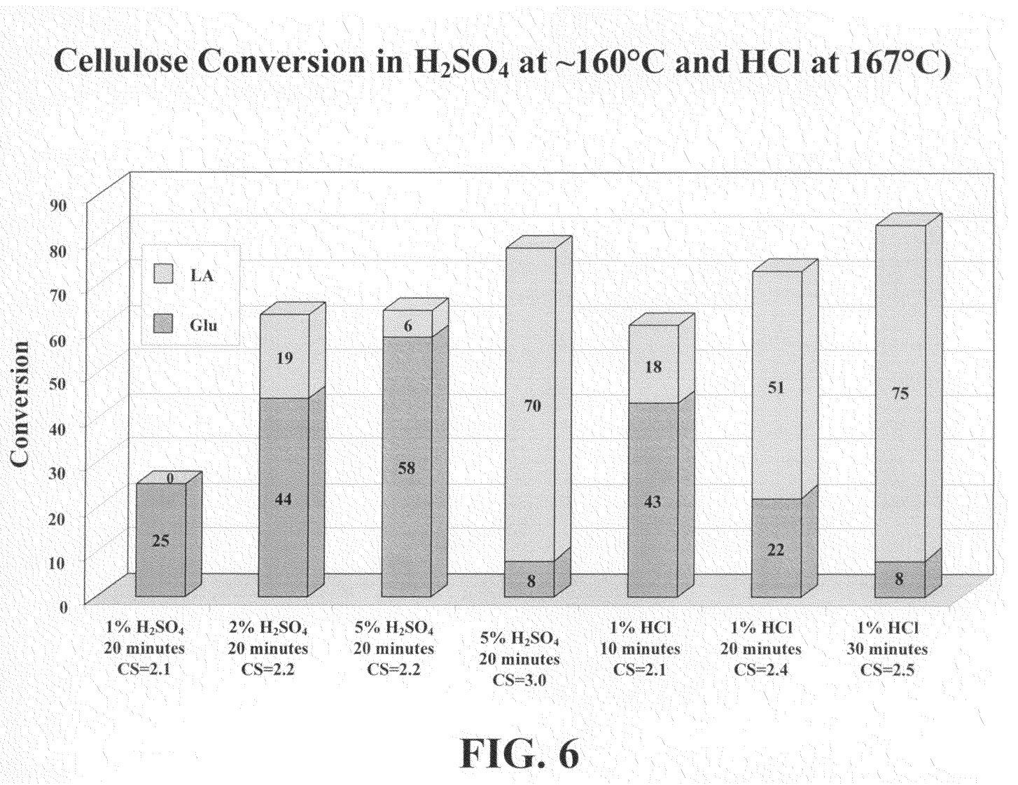

Referring to FIG. 6, the effects of cellulose conversion using microwave irradiation in the presence of sulfuric acid and hydrochloride acid at different concentrations, different times and different CS values was studied. The data shows that at twenty minutes, sulfuric acid and hydrochloride acid show similar conversions, but by controlling the time and temperature of hydrochloric acid promoted microwave digestion of cellulose, the type of products produced can be changed significantly. This is not the case with sulfuric acid.

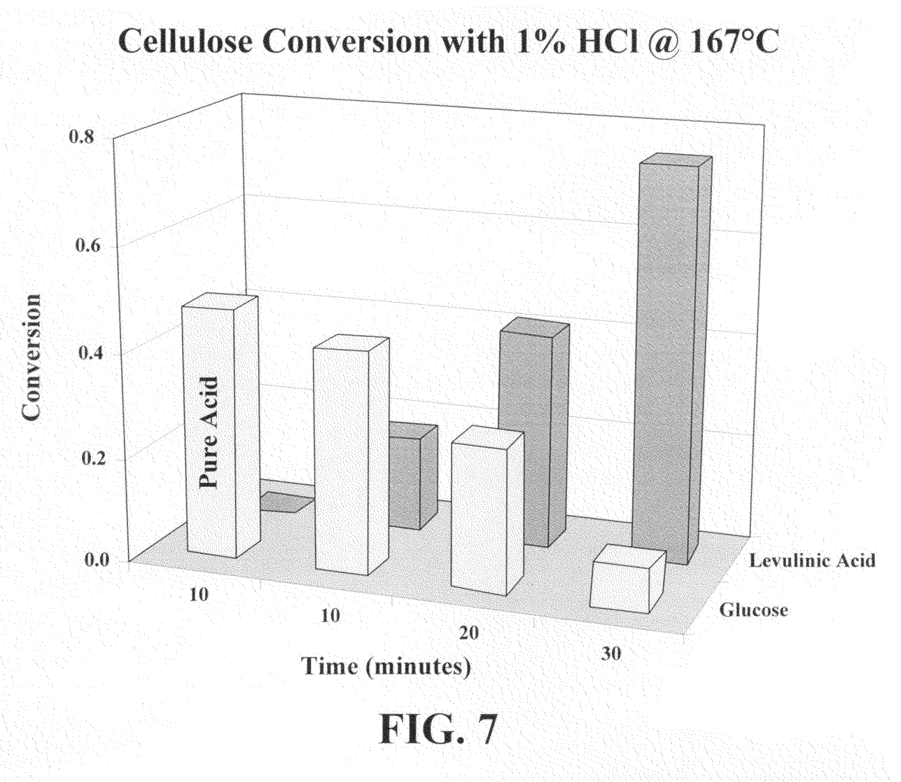

Referring to FIG. 7, the product distribution for cellulose conversion using microwave irradiation in the presence of hydrochloric acid is shown. At short time and using pure hydrochloric acid (freshly made or stored in a plastic container), the conversion yield almost exclusively glucose, while at longer time (30 minutes or more), the product is switched to levulinic acid.

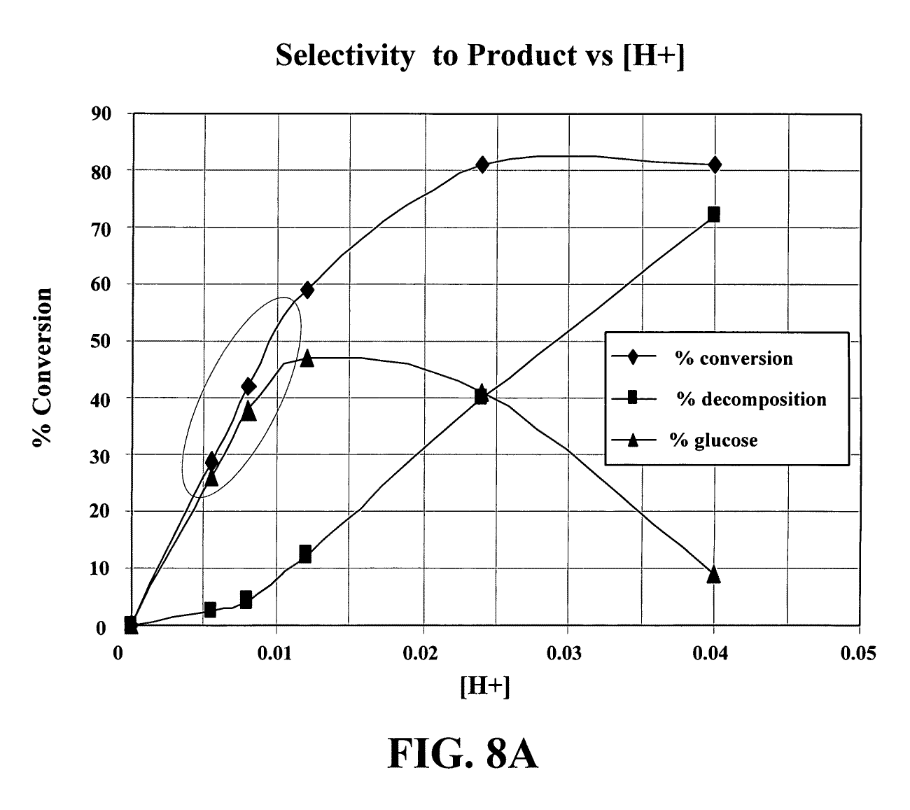

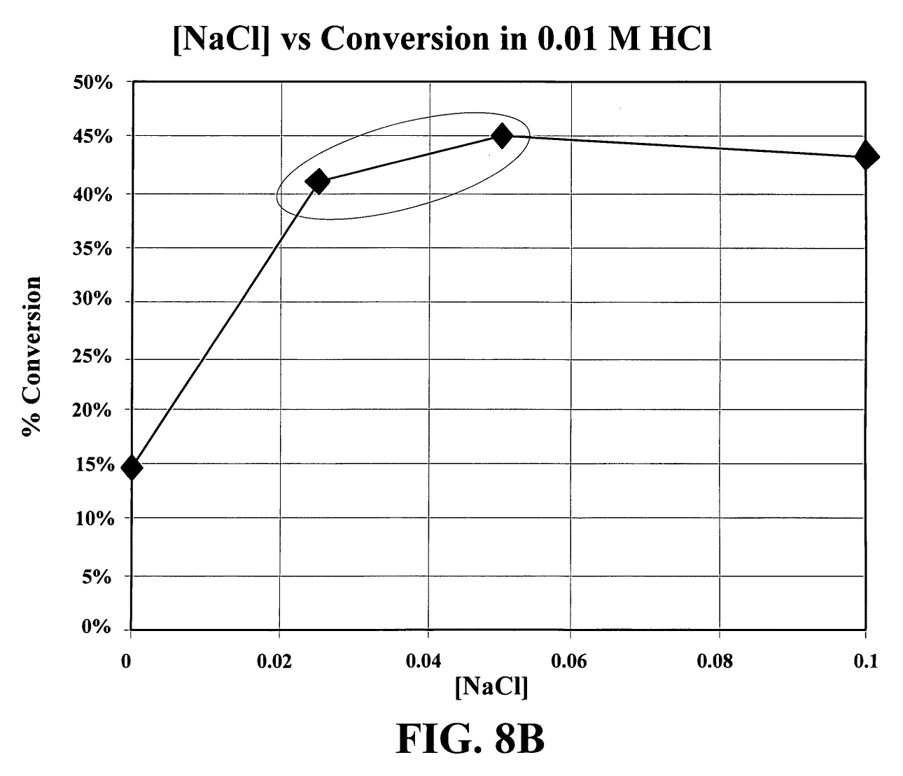

Referring to FIG. 8A, cellulose conversion and product selectivity at different hydrochloric acid concentrations was studied. The data shows that glucose conversion is maximized at acid concentration between about 0.005 and about 0.025. The data also shows that cellulose decomposition increases with increasing acid concentration with the two curves crossing at about 0.024 acid concentration. This means that by running the reaction at low acid concentration, low metal salt concentration and short time, the system can be tuned to maximize glucose production. As stated herein, the method can be designed to recycle cellulose feedstock so that each irradiation occurs for a short duration. The circled conversion range also yield high concentrations of 5-hydroxymethyl furfural. In FIG. 8B, the conversion of cellulose using microwave irradiation in the present of acid and sodium chloride. The data shows that at concentrations of sodium chloride between about 0.01 and about 0.06 is all that is required to maximize the conversion. The circled region represents a region that yields high concentrations of 5-hydroxymethyl furfural.

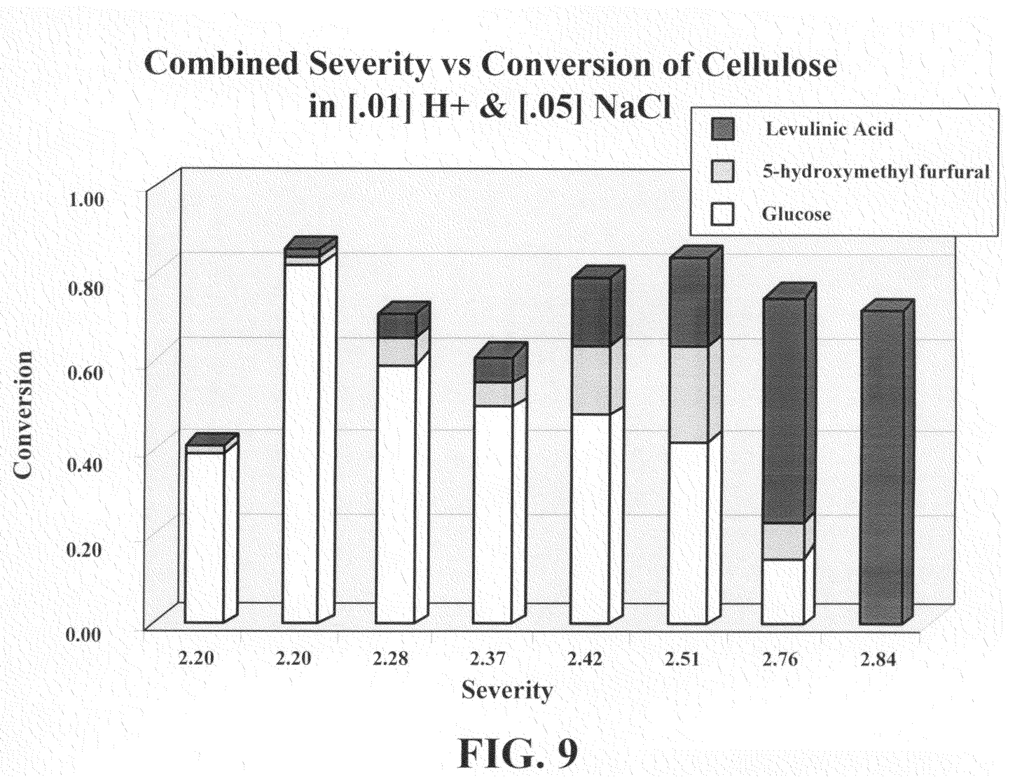

Referring to FIG. 9, the effect of reaction severity on product distribution for cellulose digesting using microwave irradiation in the presence of 0.01 M hydrochloric acid and 0.05 M NaCl was studied. The data shows that by changing the severity of the reaction, the product distribution can be radically changed. Thus, the reaction can be tuned to generated a desired product or product mix.

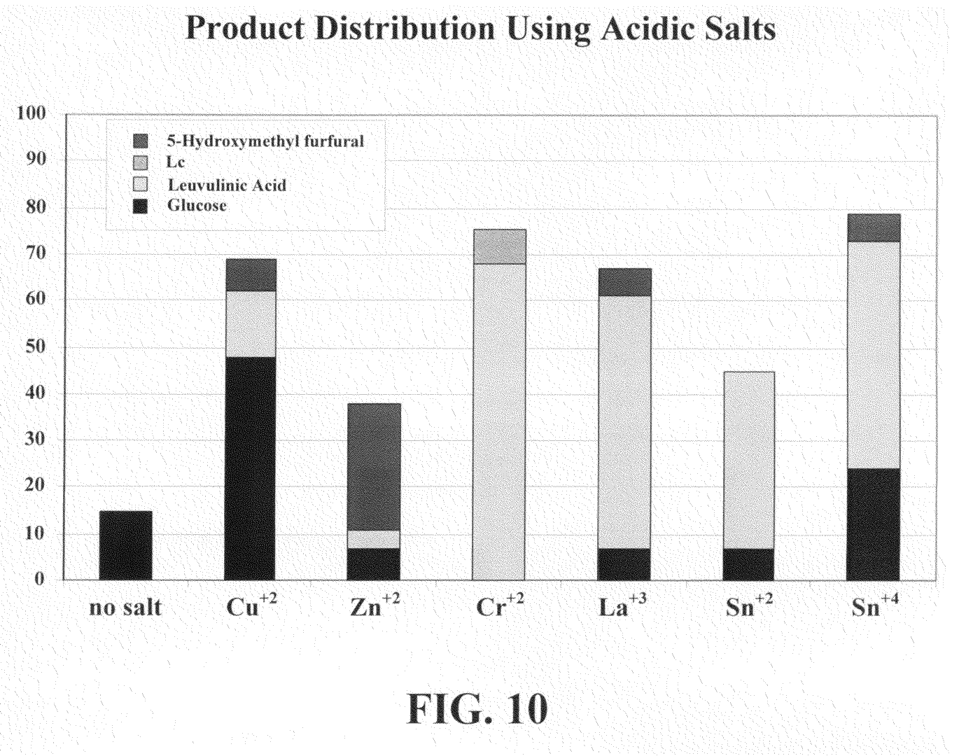

Referring to FIG. 10, the effect on product distribution was studied for cellulose digestion using microwave irradiation in the presence of pure hydrochloric acid and various transition metal. The data showed that the presence of a transition metal salt significantly increases conversion. The data also showed that different metal salts produce different product distributions. The data clearly demonstrates that different metal give different product distributions. Thus, by careful control of the reaction conditions, acid concentration, metal salt concentrations, metal salt type, temperature, pressures and time not only can conversion be maximized, but the reaction can be tuned to generated a desired product distribution. If a product mix is desired, then the various products can be separated using well known separation techniques.

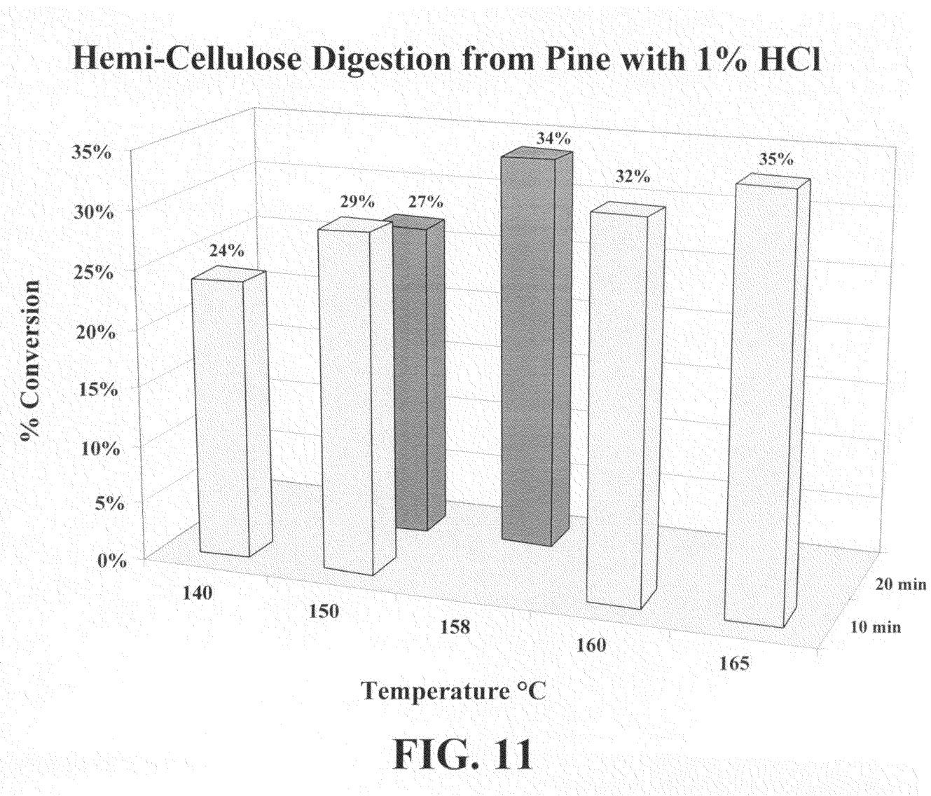

Referring to FIG. 11, the digestion of hemi-cellulose in a pine feedstock having a particle size of about 60 mesh using microwave irradiation in the presence of hydrochloric acid and a copper (II) salt at various temperatures and two times. The data showed that conversion can be maximized through the control of temperature and time.

The feedstock is irradiated in the presence of an aqueous solution including a dilute acid and a dilute metal salt. The feedstock generally comprises saccharide-containing material. In certain embodiments, the feedstock comprises a cellulosic material having a low amount of lignins. In general suitable feedstocks include plant wastes, which are mostly cellulose and hemicellulose, such as plant stems, leaves, stalks, stover, husks, hulls, waste paper, and the like or mixtures or combinations thereof. Suitable acids for use in the present invention include, without limitation, mineral acids, organic acids and mixtures or combinations thereof. Suitable mineral acids include, without limitation, sulfuric acid, hydrochloric acid, phosphoric acid, or other mineral acids or mixtures or combinations thereof. Examples of organic acids include, without limitation, acetic, citric, tartaric, formic, propanoic, propenoic, and the like. In certain embodiments, the acid is a mineral acid. In most embodiments, the acid is hydrochloric acid. In other embodiments, the mineral acid is pure hydrochloric acid stored in plastic containers or other containers that are not ceramic or glass in nature to reduce contamination by detrimental salts that are leached from the ceramic or glass by action of the acid. Suitable metal salts include, without limitation, alkali metal salts, alkaline earth metal salts, transition metal salts or mixtures or combinations thereof. In certain embodiments, the transition metal salts include Cu⁺² salts, Zn⁻² salts, Cr⁺² salts, Cr⁺³ salts, La⁺³ salts, Sn^{|2} salts, Sn^{|4} salts, Fe^{|2} salts, Fe^{|3} salts, Co^{|2} salts. Exemplary transition metal salts include ZnCl₂, ZnSO₄, CuCl₂, FeCl₃, FeSO₄, CrSO₄, CrCl₃, CuSO₄, CoCl₂, CoSO₄, LaCl₃ and mixture or combinations thereof. In most embodiments, the metal salts are chloride metal salts.

TABLE 1
Experimental Results for Substrate Conversion

| Substrate | Substrate % | Acid | M | Salt | M | ° C. | min | Solubles % | HMF* % | LA % | Glu % | Yield % |
|---|---|---|---|---|---|---|---|---|---|---|---|---|
| starch | 10 | HCl | 0.004 | NaCl | | | | | | | | |
| starch | 10 | HCl | 0.004 | ZnCl₂ | 0.03 | 196 | 10 | 98 | 34 | 0.5 | 63.5 | 98 |
| starch | 10 | HCl | 0.004 | ZnCl₂ | 0.67 | 197 | 10 | 82 | 57 | 11 | 14 | 82 |
| starch | 10 | HCl | 0.004 | ZnCl₂ | 1.33 | 197 | 5 | 86 | 49 | 6 | 31 | 86 |
| starch | 10 | HCl | 0.160 | ZnCl₂ | 0.17 | 180 | 15 | 88 | 0 | 69 | 19 | 88 |
| starch | 20 | HCl | 0.004 | ZnCl₂ | 0.67 | 197 | 5 | 85 | 36 | 7 | 42 | 85 |
| glucose | 20 | HCl | 0.004 | ZnCl₂ | 0.67 | 197 | 5 | 79 | 39 | 10 | 30 | 79 |
| glucose | 20 | HCl | 0.004 | ZnCl₂ | 0.17 | 196 | 5 | 93 | 25 | 4 | 64 | 93 |
| sucrose | 20 | HCl | 0.004 | ZnCl₂ | 0.17 | 196 | 5 | 79 | 0 | 71 | 8 | 79 |
| sucrose | 20 | HCl | 0.004 | ZnCl₂ | 0.67 | 196 | 5 | 75 | 30 | 13 | 32 | 75 |
| starch | 10 | HCl | 0.004 | InCl₂ | 0.04 | 196 | 10 | 85 | 47 | 28 | 10 | 85 |
| starch | 10 | HCl | 0.060 | CrCl₂ | 0.04 | 180 | 5 | 90 | 45 | 40 | 5 | 90 |
| starch | 10 | HCl | 0.016 | Al₂(SO₄)₃ | 0.04 | 180 | 5 | 88 | 57 | 4 | 27 | 88 |
| starch | 10 | H₂SO₄ | 0.330 | — | 0.0 | 196 | 5 | 81 | 0 | 81 | 0 | 81 |

*Formic Acid is co-produced in the same amount.
M = molarity
HMF = 5-hydroxymethyl-2-furfural
LA = Levulinic acid
Glu = Glucose
% HMF, % LA, % Glu are based on the theoretical yields and are estimated by 13C NMR spectra of the filtered salt water solutions.

5-hydroxymethyl-2-furfural and Levulinic acid can be isolated from the recycled aqueous solution by conventional extraction as well as by ion exchange or absorption techniques and confirmed the NMR estimates.

The glucose remaining in the recycled solution continues to react with each pass. FeCl₃, CrCl₃, CuSO₄, CoCl₂ and LaCl₃ all gave similar, but less favorable results under the same range of conditions.

Cascade Reactions to Digest Remaining Cellulose from Pine Residue

Pine feedstock comprising 29% ligin, ˜32% hemicellulose, and 39% cellulose including about 71% sugar or saccharide units. Three (3) sequential irradiations with microwave energy in the solution of a dilute acid and a dilute metal salt at 208° C. for 10 minutes resulted in a 72% dissolution ofthe feedstock with ˜20% decomposition of cellulose. Three (3) sequential irradiations with microwave energy in the solution of a dilute acid and a dilute metal salt resulted in a reaction temperature of spike up to 230° C. and reaction temperature of 198° C. for 10 minutes resulted in ˜71% dissolution of the feedwook with ˜5% decomposition of cellulose. The feedstock was loaded at about a 12% load comprising 60 mesh particles. The sequential reaction produced about 4% xylose and about 4% glucose, 3% glucose and 2% glucose in each irradiation. Approximate theoretical conversion of all carbohydrates to usable aldose platforms.

Advantages of the methods of this invention include: (1) higher efficiencies (high conversions to desired products), (2) shorter reaction times (10-100 times faster than conventional reactions) and (3) smaller amounts of reagents. The method can be used to produce platform chemicals including xylose (using hemicellulose mix), glucose, levulinic acid, and 5-hydromethyl furfural.

The acid is used in a mass ratio of from about 1 to about 10 per unit of cellulose-based matter. In certain embodiments, the mass ratio is between about 1 and about 5 per unit of cellulose-based matter. Water is used in a mass ratio of from about 1 to about 10 per unit of cellulose-based matter. The mass ratio of acid to water is from about 0.1:1 to about 10:1. In certain embodiments, the mass ratio is between about 0.5:1 and about 2:1.

A process and associated apparatus for converting a saccharide-containing feedstock such as glucose, sucrose, cellulose, hemi-cellulose, cellulosic waste (biomass), or mixtures thereof into soluble saccharide products and/or a variety of platform chemicals has been developed. The basic method includes irradiating an aqueous saccharide-containing feedstock with microwave radiation in the presence of a dilute acid and a dilute metal salt for relatively short times, at elevated temperatures or at elevated temperatures and pressures. Under these conditions, the saccharide-containing feedstock is efficiently hydrolyzed to desired saccharide or platform chemicals.

### Embodiments of Apparatus for Use in the Performance of the Methods of This Invention

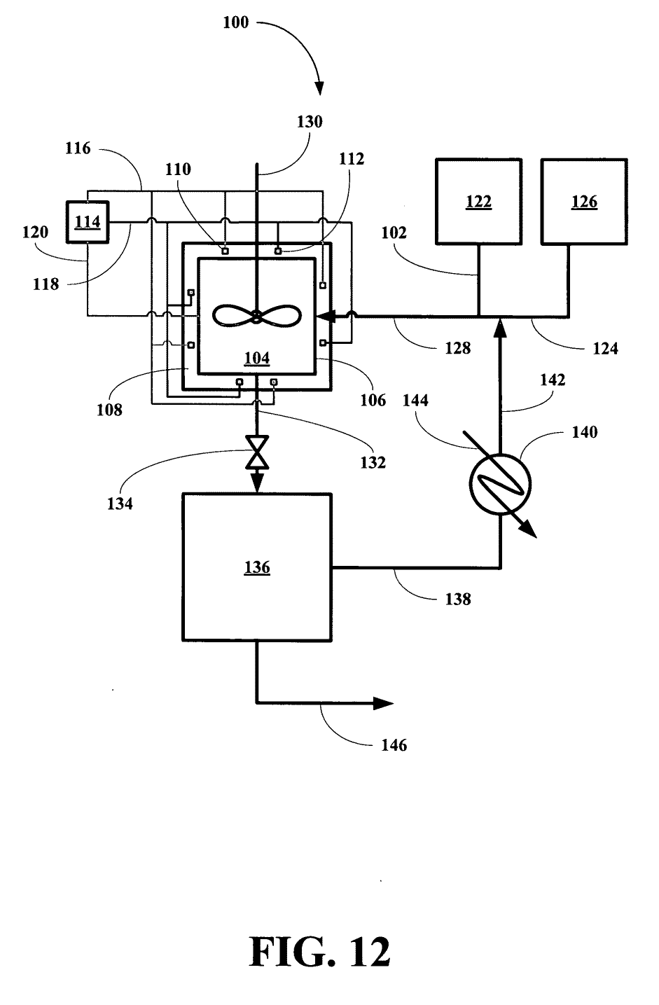

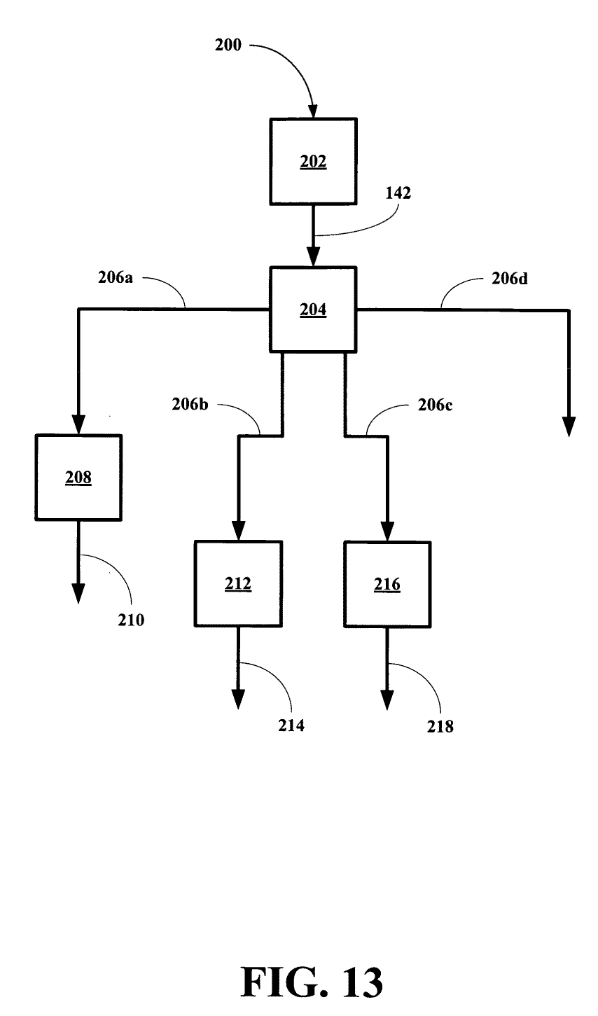

Referring to FIG. 12, an apparatus, generally 100, for converting a saccharide-containing feedstock into low molecular products is shown. The apparatus 100 of the present invention is designed to irradiate an aqueous saccharide-containing feedstock stream 102 in the presence of an acidic metal salt solution. The reactor 104 comprises a reactor Multi-dimensional Analysis of Reactor Safety (MARS) reactor using a CEM microwave source associated with the reactor 104. The reactor 104 comprises a high pressure XP1500 teflon vessel 106 having a kevlar shield 108 fitted with both pressure sensors 110 and temperatures sensors 112, which in turn are used to control the programmed set point and adjust the power accordingly. Typically, the pressure sensors 110 and the temperature sensors 112 are connected to a programmable electronic device 114 such as a microprocessor, which senses the pressure and temperature in the reactor 104 via pressures sensor signals 116 and temperature senor signals 118. The signals 116 and 118 are used by the device 114 to control the CEM microwave generator associated with the reactor 104 via a power control line 120. CEM microwave reactors are available from CEM Corporation of Matthew, N.C., USA. Additionally information on microwave reactors can be found in U.S. Pat. Nos. 5,206,479, 5,318,754, and 6,344,120, incorporated herein by reference.

The feedstock stream 102 is feed to the reactor 104 from a feedstock reservoir 122. The feedstock stream 102 is mixed with an aqueous stream 124 including a dilute acid and a dilute metal salt from a acidic metal salt solution reservoir 126. The apparatus 100 is shown with the two stream 102 and 112 being mixed to form a mixed stream 128 prior to entering the reactor 104, but the two stream 102 and 124 can be fed separating to the reactor prior to irradiating the feedstock with the microwave radiation in the reactor 104.

The reactor 104 is provided with an agitator 130.

The reactor 104 is generally operated at a controlled equilibrium pressure in a range of from about 2 atm to about 20 atm. In certain embodiments, the pressure is in the range between about 4 atm and about 10 atm. The upper limit on the operating pressure is set by the design requirements of the reactor 104, the power requirements of the microwave source 106, and possibly the maximum temperature tolerated by the feed material without decomposition into undesired carbonaceous matter. Reactor temperature generally corresponds to the saturation temperature. The time length of the pretreatment P at the equilibrium pressure should be sufficient to effect enhancement of the subsequent hydrolysis rate. Typically, the feedstock stream 102 is irradiated for a time period of from about 1 minute to about 30 minutes. In certain embodiments, the pretreatment is between about 2 and about 30 minutes. In other embodiments, the pretreatment is between about 5 and about 30 minutes. In other embodiments, the pretreatment is between about 5 and about 20 minutes. In other embodiments, the pretreatment is between about 10 and about 20 minutes. The microwave radiation can also be pulsed to further modify the reaction conditions.

A slurry effluent stream 132 exiting the reactor 104 is passed through the depressurization valve 134 into a separation vessel 136 wherein solids and liquids are separated. Optionally, a stream 138 comprising a recycle acidic metal salt solution passes into a condenser 140, where it is fully condensed to form a fully condensed acidic metal salt solution stream 142, which can be added into the stream 112 prior to or after mixing with the stream 102. Means for separating a liquid and solid are well known to include filtration, decanting, centrifuging, evaporation, and the like. The vessel 132 can include a heater (not shown) to enhance the separation of water, acid and metal salt components. The condenser 136 uses an heat exchange medium 144 such as cooling water to condense the stream 128. The condensed stream 138 is then available for reuse in the autoclave 104. Prior to use, the recycle stream 138 can be retained in a storage vessel (not shown) until needed. A product stream 146 is produced, which can be further process down stream.

Following the partial or complete conversion of the polysaccharides in the feedstock into soluble products, a reaction effluent stream 146 can be further processed. Referring now to FIG. 13, an apparatus, generally 200, is shown to including the apparatus 100, represented by the box 202 and a separation unit 204, where the stream 142 is separated into a plurality of product stream 206a-d. If the product stream 206a comprises a glucose rich product, then the stream 206a can be forwarded to a fermentation system 208, where it is fermented into a biofuel containing ethanol stream 210. If the product stream 206b comprises a levulinic acid rich product, then the stream 206b can be sent to a product purification unit 212, where purified levulinic acid stream 214 is produced. If the product stream 206c comprises a 5-hydroxymethyl furfural rich product, then the stream 206c can be sent to a product purification unit 216, where purified 5-hydroxymethyl furfural stream 218 is produced. The stream 206d can comprises a stream of other products.

All references cited herein are incorporated by reference Although the invention has been disclosed with reference to its preferred embodiments, from reading this description those of skill in the art may appreciate changes and modification that may be made which do not depart from the scope and spirit of the invention as described above and claimed hereafter.

I claim:

1. A method of digesting a saccharide-containing feedstock comprising the steps of:
treating a saccharide-containing feedstock in a dilute solution including an acid and a metal salt in the presence of microwave energy,
where an amount of the metal salt, an amount of the acid and an amount of the microwave energy are sufficient to convert a desired amount of the polymer to useable platform chemicals at a temperature between about 150° C. and about 250° C. and for a time between about 1 minute and about 30 minutes.

2. The method of claim 1, wherein the feedstock comprises saccharide-containing material.

3. The method of claim 1, wherein the feedstock comprises a cellulosic material having a low amount of lignins.

4. The method of claim 1, wherein the feedstock comprises a plant waste.

5. The method of claim 1, wherein the plant waste is selected from the group consisting of plant stems, leaves, stalks, stover, husks, hulls, waste paper, and the like or mixtures or combinations thereof.

6. The method of claim 1, wherein the acid is selected from the group consisting of mineral acids, organic acids and mixtures or combinations thereof.

7. The method of claim 1, wherein the mineral acids are selected from the group consisting of sulfuric acid, hydrochloric acid, phosphoric acid, or other mineral acids or mixtures or combinations thereof.

8. The method of claim 1, wherein the acid is hydrochloric acid.

9. The method of claim 1, wherein the acid is pure hydrochloric acid stored in plastic containers or other containers that are not ceramic or glass in nature to reduce contamination by detrimental salts that are leached from the ceramic or glass by action of the acid.

10. The method of claim 1, wherein the metal salt is selected from the group consisting of alkali metal salts, alkaline earth metal salts, transition metal salts or mixtures or combinations thereof.

11. The method of claim 1, wherein the metal salt is a transition metal salt or mixtures thereof.

12. The method of claim 1, wherein the metal salt is selected from the group consisting of Cu⁺² salts, Zn⁺² salts, Cr⁺² salts, Cr⁺³ salts, La⁺³ salts, Sn⁺² salts, Sn⁺⁴ salts, Fe⁺² salts, Fe⁺³ salts, Co⁺² salts and mixtures or combinations thereof.

13. The method of claim 1, wherein the metal salt is selected from the group consisting of ZnCl₂, ZnSO₄, CuCl₂, FeCl₃, FeSO₄, CrSO₄, CrCl₃, CuSO₄, CoCl₂, CoSO₄, LaCl₃ and mixture or combinations thereof.

14. The method of claim 1, wherein the feedstock is mechanically milled.

15. The method of claim 1, further comprising the step of;
fermenting a glucose rich product into an ethanol containing bio-fuel.

16. A method for generating a bio-fuel comprising the steps of:
digesting a saccharide-containing feedstock in a dilute solution including an acid and a metal salt in the presence of a microwave energy, where an amount of metal salt, an amount of the acid and an amount of the microwave energy are sufficient to convert a desired amount of the polymer to glucose at a temperature between about 150° C. and about 250° C. and for a time between about 1 minute and about 30 minutes, and
fermenting the glucose into a ethanol containing bio-fuel.

17. The method of claim 16, wherein the feedstock is selected from the group consisting of plant stems, leaves, stalks, stover, husks, hulls, waste paper, and the like or mixtures or combinations thereof.

18. The method of claim 16, wherein the acid is selected from the group consisting of mineral acids, organic acids and mixtures or combinations thereof.

19. The method of claim 16, wherein the mineral acids are selected from the group consisting of sulfuric acid, hydrochloric acid, phosphoric acid, or other mineral acids or mixtures or combinations thereof.

20. The method of claim 16, wherein the acid is hydrochloric acid.

21. The method of claim 16, wherein the acid is pure hydrochloric acid stored in plastic containers or other containers that are not ceramic or glass in nature to reduce contamination by detrimental salts that are leached from the ceramic or glass by action of the acid.

22. The method of claim 16, wherein the metal salt is selected from the group consisting of alkali metal salts, alkaline earth metal salts, transition metal salts or mixtures or combinations thereof.

23. The method of claim 16, wherein the metal salt is a transition metal salt or mixtures thereof.

24. The method of claim 16, wherein the metal salt is selected from the group consisting of Cu⁺² salts, Zn⁺² salts, Cr⁺² salts, Cr⁺³ salts, La⁺³ salts, Sn⁺² salts, Sn⁺⁴ salts, Fe⁺² salts, Fe⁺³ salts, Co⁺² salts and mixtures or combinations thereof.

25. The method of claim 16, wherein the metal salt is selected from the group consisting of ZnCl₂, ZnSO₄, CuCl₂, FeCl₃, FeSO₄, CrSO₄, CrCl₃, CuSO₄, CoCl₂, CoSO₄, LaCl₃ and mixture or combinations thereof.

26. The method of claim 16, wherein the feedstock is mechanically milled.

27. The method of claim 1, further comprising a plurality of digesting steps.

28. The method of claim 16, further comprising a plurality of digesting steps.
